(19) United States
(12) Patent Application Publication
(10) Pub. No.: US 2010/0159009 A1
(43) Pub. Date: Jun. 24, 2010

(54) CONTROLLED-RELEASE FORMULATIONS

(76) Inventors: Zhongshui Yu, Bridgewater, NJ (US); Kristin Arnold, Morrisville, PA (US); Siva Rama K. Nutalapati, Princeton, NJ (US)

Correspondence Address: CANTOR COLBURN, LLP, 20 Church Street, 22nd Floor, Hartford, CT, 06103, US

(21) Appl. No.: 12/641,377

(22) Filed: Dec. 18, 2009

Related U.S. Application Data
(60) Provisional application No. 61/140,722, filed on Dec. 24, 2008.
(60) Provisional application No. 61/168,698, filed on Apr. 13, 2009.

(51) Int. Cl. A61K 31/40; A61K 9/00

(52) U.S. Cl. 424/484; 514/424

(57) ABSTRACT

Disclosed herein are extended-release levetiracetam formulations having a matrix comprising levetiracetam and a hydrophobic excipient or an acrylic polymer excipient.

### CROSS REFERENCE TO RELATED APPLICATION

This application claims the benefit of U.S. Provisional Application Ser. Nos. 61/140,722 filed Dec. 24, 2008 and 61/168,698 filed Apr. 13, 2009, which are hereby incorporated by reference in their entirety.

### BACKGROUND

Controlled-release dosage formulations, including sustained-release formulations, provide a variety of benefits to the patient such as reduction in the number of doses per day, increased convenience, reduced occurrences of missed doses, and the chance to achieve controlled blood levels of the active agent.

Levetiracetam, a single enantiomer, (−)-(S)-α-ethyl-2-oxo-1-pyrrolidine is used for adjunctive therapy in treatment of partial onset seizures in patients with or without epilepsy.

An immediate-release tablet containing 250 mg, 500 mg, 750 mg or 1000 mg levetiracetam is currently commercially marketed in the United States. The tablets are administered orally to a patient twice-daily to reach a cumulative daily target of up to 3000 mg per day. Also currently available is a once-daily levetiracetam tablet containing 500 mg or 750 mg levetiracetam.

There remains a need, however, for improved oral pharmaceutical formulations for the controlled release of active agents such as levetiracetam to allow for reduced incidents of administration, specifically single daily dose administrations. Also needed are dosage formulations having substantially no food effect such that a patient has the convenience of taking the dosage formulation with or without food.

### SUMMARY

In one embodiment, an extended-release formulation comprises a matrix comprising levetiracetam or a pharmaceutically acceptable salt, solvate, hydrate, crystalline form or non-crystalline form thereof, and a hydrophobic excipient or an acrylic polymer excipient; wherein the extended-release formulation is substantially free of an extended-release coating.

In another embodiment, an extended-release formulation comprises a matrix comprising levetiracetam or a pharmaceutically acceptable salt, solvate, hydrate, crystalline form or non-crystalline form thereof, and a hydrophobic excipient or an acrylic polymer excipient; wherein the extended-release formulation is free of an extended-release coating.

In another embodiment, an extended-release formulation comprises a matrix comprising levetiracetam or a pharmaceutically acceptable salt, solvate, hydrate, crystalline form or non-crystalline form thereof, and a hydrophobic excipient or an acrylic polymer excipient; wherein the extended-release formulation is substantially free of an extended-release coating, wherein the extended-release formulation is bioequivalent to a reference drug according to New Drug Application No. 022285 when administered to a patient in a fasted or non-fasted state.

In yet another embodiment, an extended-release formulation comprises a matrix comprising levetiracetam or a pharmaceutically acceptable salt, solvate, hydrate, crystalline form or non-crystalline form thereof, and a hydrophobic excipient or an acrylic polymer excipient; wherein the extended-release formulation is substantially free of an extended-release coating, wherein the extended release formulation exhibits substantially no food effect.

In one embodiment, an extended-release formulation comprises a matrix comprising levetiracetam or a pharmaceutically acceptable salt, solvate, hydrate, crystalline form or non-crystalline form thereof, about 15 to about 25 weight percent carnauba wax based on the total weight of the matrix, and about 5 to about 15 weight percent stearic acid based on the total weight of the matrix; wherein the extended-release formulation is substantially free of an extended-release coating.

These and other embodiments, advantages and features of the present invention become clear when detailed description and examples are provided in subsequent sections.

### DETAILED DESCRIPTION

Disclosed herein are extended-release formulations comprising a matrix of levetiracetam or a pharmaceutically acceptable salt, solvate, hydrate, crystalline form or non-crystalline form thereof, and a hydrophobic excipient or an acrylic polymer excipient; wherein the extended-release formulation is substantially free of an extended-release coating. Further embodiments include extended-release formulations comprising a matrix of levetiracetam or a pharmaceutically acceptable salt, solvate, hydrate, crystalline form or non-crystalline form thereof, and a hydrophobic excipient or an acrylic polymer excipient, wherein the matrix is substantially free of a hydrophilic polymeric excipient; and wherein the extended-release formulation is substantially free of an extended-release coating.

The terms “a” and “an” do not denote a limitation of quantity, but rather denote the presence of at least one of the referenced item. The term “or” means “and/or”. The terms “comprising”, “having”, “including”, and “containing” are to be construed as open-ended terms (i.e., meaning “including, but not limited to”). The endpoints of all ranges directed to the same component or property are inclusive and independently combinable.

An “active agent” means a compound, element, or mixture that when administered to a patient, alone or in combination with another compound, element, or mixture, confers, directly or indirectly, a physiological effect on the patient. The indirect physiological effect may occur via a metabolite or other indirect mechanism. When the active agent is a compound, then salts, solvates (including hydrates) of the free compound or salt, crystalline forms, non-crystalline forms, and any polymorphs of the compound are contemplated herein. Compounds may contain one or more asymmetric elements such as stereogenic centers, stereogenic axes and the like, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In addition, compounds with carbon-carbon double bonds may occur in Z- and E-forms, with all isomeric forms of the compounds. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

“Pharmaceutically acceptable salts” includes derivatives of the active agent, wherein the active agent is modified by making acid or base addition salts thereof, and further refers to pharmaceutically acceptable solvates, including hydrates, crystalline forms, and non-crystalline forms of such salts. Examples of pharmaceutically acceptable salts include, but are not limited to, mineral or organic acid addition salts of basic residues such as amines; alkali or organic addition salts of acidic residues; and the like, and a combination comprising at least one of the foregoing salts. The pharmaceutically acceptable salts include salts and the quaternary ammonium salts of the active agent. For example, acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, and a combination comprising at least one of the foregoing salts. Pharmaceutically acceptable organic salts includes salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparginate, glutamate, and the like; and a combination comprising at least one of the foregoing salts.

“Levetiracetam” means levetiracetam or a pharmaceutically acceptable levetiracetam salt, including any solvate, hydrate, crystalline form, and non-crystalline form thereof unless otherwise indicated.

“Reference drug” means a levetiracetam product as described in U.S. Federal Food and Drug Administration's New Drug Application No. 022285 approved on Sep. 12, 2008 (500 mg) or Feb. 12, 2009 (750 mg) as provided in the U.S. Federal Food and Drug Administration's Orange Book, Approved Drug Products with Therapeutic Equivalence Evaluations. Keppra XR™ is a levetiracetam oral, extended-release tablet product available in 500 mg and 750 mg strengths. Keppra XR™, 750 mg is the “reference listed drug” under 21 CFR 314.94(a)(3)), i.e., the listed drug identified by FDA as the drug product upon which an applicant relies in seeking approval of its ANDA.

A “dosage form” or “dosage formulation” means a unit of administration of an active agent. Examples of dosage formulations include tablets, capsules, injections, suspensions, liquids, emulsions, creams, ointments, suppositories, inhalable formulations, transdermal formulations, and the like. “Form” and “formulation” are to be used interchangeably unless indicated otherwise.

By “oral dosage form” is meant to include a unit dosage form for oral administration. An oral dosage form may optionally comprise a plurality of subunits such as, for example, microcapsules or microtablets. Multiple subunits may be packaged for administration in a single dose.

By “subunit” is meant to include a composition, mixture, particle, pellet, and the like, that can provide an oral dosage form alone or when combined with other subunits.

“Bioavailability” means the extent or rate at which an active agent is absorbed into a living system or is made available at the site of physiological activity. For active agents that are intended to be absorbed into the bloodstream, bioavailability data for a given formulation may provide an estimate of the relative fraction of the administered dose that is absorbed into the systemic circulation. “Bioavailability” can be characterized by one or more pharmacokinetic parameters.

“Pharmacokinetic parameters” describe the in vivo characteristics of an active agent (or surrogate marker for the active agent) over time, such as plasma concentration (C), Cₘₐₓ, Cₙ, C₂₄, Tₘₐₓ, and AUC. “Cₘₐₓ” is the measured concentration of the active agent in the plasma at the point of maximum concentration. “Cₙ” is the measured concentration of an active agent in the plasma at about n hours after administration. “C₂₄” is the measured concentration of an active agent in the plasma at about 24 hours after administration. The term “Tₘₐₓ” refers to the time at which the measured concentration of an active agent in the plasma is the highest after administration of the active agent. “AUC” is the area under the curve of a graph of the measured concentration of an active agent (typically plasma concentration) vs. time, measured from one time point to another time point. For example AUC₀₋ₜ is the area under the curve of plasma concentration versus time from time 0 to time t. The AUC_{0-∞} or AUC_{0-INF} is the calculated area under the curve of plasma concentration versus time from time 0 to time infinity.

“Food” typically means a solid food or mixed solid/liquid food with sufficient bulk and fat content that it is not rapidly dissolved and absorbed in the stomach. In one embodiment, food means a meal, such as breakfast, lunch or dinner. The terms “taken with food”, “fed” and “non-fasted” are equivalent and are as given by FDA guidelines and criteria. In one embodiment, with food means that the dosage form is administered to a patient between about 30 minutes prior to about 2 hours after eating a meal. In another embodiment, with food means that the dosage form is administered at substantially the same time as the eating the meal.

The terms “without food”, “fasted” and “an empty stomach” are equivalent and are as given by FDA guidelines and criteria. In one embodiment, fasted is means the condition wherein no food is consumed within 1 hour prior to administration of the dosage form or 2 hours after administration of the dosage form. In another embodiment, fasted means the condition wherein no food is consumed within 1 hour prior to administration of the dosage form to 2 hours after administration of the dosage form.

“Substantially no food effect” means that the pharmacokinetics are substantially the same for the oral administration of the formulation under fed conditions (“non-fasting”) when compared to administration under fasting conditions. For example, the comparison between Cₘₐₓ or AUC of a single administration of a formulation under fed conditions to a single administration of the same formulation under fasted conditions results in a percent ratio of Cₘₐₓ or AUC having a 90% confidence interval upper limit of less than or equal to 125% or a lower limit of greater than or equal to 80%. Such information can be based on logarithmic transformed data. Exemplary study considerations can be found in the Federal Drug Administration's (FDA) guidelines and criteria, including “Guidance for Industry, Food-Effect Bioavailability and Fed Bioequivalence Studies” available from the U.S. Department of Health and Human Services (DHHS), Food and Drug Administration (FDA), Center for Drug Evaluation and Research (CDER) December 2002, incorporated herein in its entirety.

A dissolution profile is a plot of the cumulative amount of active agent released from a formulation as a function of time. A dissolution profile can be measured utilizing the Drug Release Test <724>, which incorporates standard test USP 26 or 28 (Test <711>), incorporated herein by reference in its entirety. A profile is characterized by the test conditions selected such as, for example, apparatus type, shaft speed, temperature, volume, and pH of the dissolution medium. More than one dissolution profile may be measured. For example, a first dissolution profile can be measured at a pH level approximating that of the stomach, and a second dissolution profile can be measured at a pH level approximating that of one point in the intestine or several pH levels approximating multiple points in the intestine.

A highly acidic pH may be employed to simulate the stomach and a less acidic to basic pH may be employed to simulate the intestine. By the term “highly acidic pH” is meant a pH of about 1 to about 4. A pH of about 1.2, for example, can be used to simulate the pH of the stomach. By the term “less acidic to basic pH” is meant a pH of greater than about 4 to about 7.5, specifically about 6 to about 7.5. A pH of about 6 to about 7.5, specifically about 6.8, can be used to simulate the pH of the intestine.

By “immediate-release” is meant a conventional or non-modified release in which greater then or equal to about 75% of the active agent is released within two hours of administration, specifically within one hour of administration.

By “controlled-release” is meant a dosage form in which the release of the active agent is controlled or modified over a period of time. Controlled can mean, for example, extended-, sustained-, delayed- or pulsed-release at a particular time. Alternatively, controlled can mean that the release of the active agent is extended for longer than it would be in an immediate-release dosage form, e.g., at least over several hours.

The matrix can be formulated as a particle, a pellet, a bead, a tablet, and the like, specifically as a tablet.

In some embodiments, the formulations described herein exhibit bioequivalence to the marketed drug product, for example KEPPRA XR™ 500 mg New Drug Application no. 022285.

“Bioequivalence” means the absence of a significant difference in the rate and extent to which the active agent or surrogate marker for the active agent in pharmaceutical equivalents or pharmaceutical alternatives becomes available at the site of action when administered in an appropriately designed study.

In one embodiment, bioequivalence is any definition thereof as promulgated by the U.S. Food and Drug Administration or any successor agency thereof. In a specific embodiment, bioequivalence is determined according to the Federal Drug Administration's (FDA) guidelines and criteria, including “GUIDANCE FOR INDUSTRY BIOAVAILABILITY AND BIOEQUIVALENCE STUDIES FOR ORALLY ADMINISTERED DRUG PRODUCTS-GENERAL CONSIDERATIONS” available from the U.S. Department of Health and Human Services (DHHS), Food and Drug Administration (FDA), Center for Drug Evaluation and Research (CDER) March 2003 Revision 1; and “GUIDANCE FOR INDUSTRY STATISTICAL APPROACHES TO ESTABLISHING BIOEQUIVALENCE” DHHS, FDA, CDER, January 2001, both of which are incorporated herein in their entirety.

In an embodiment, bioequivalence of a composition to a reference drug is determined by an in vivo pharmacokinetic study to determine a pharmacokinetic parameter for the active agent composition. Specifically, bioequivalence can be determined by an in vivo pharmacokinetic study comparing a pharmacokinetic parameter for the two compositions. A pharmacokinetic parameter for the active agent composition or the reference drug can be measured in a single or multiple dose bioequivalence study using a replicate or a nonreplicate design. For example, the pharmacokinetic parameters for active agent composition of the present invention and for a reference drug can be measured in a single dose pharmacokinetic study using a two-period, two-sequence crossover design. Alternately, a four-period, replicate design crossover study may also be used. Single doses of the test composition and reference drug are administered and blood or plasma levels of the active agent are measured over time. Pharmacokinetic parameters characterizing rate and extent of active agent absorption are evaluated statistically.

The area under the plasma concentration-time curve from time zero to the time of measurement of the last quantifiable concentration (AUC₀₋ₜ) and to infinity (AUC_{0-∞}), Cₘₐₓ, and Tₘₐₓ can be determined according to standard techniques. Statistical analysis of pharmacokinetic data is performed on logarithmic transformed data (e.g., AUC₀₋ₜ, AUC_{0-∞}, or Cₘₐₓ data) using analysis of variance (ANOVA).

Under U.S. FDA guidelines, two products (e.g., an inventive levetiracetam formulation and KEPPRA XR™ 500 mg) or methods (e.g., dosing under non-fasted versus fasted conditions) are bioequivalent if the 90% Confidence Interval (CI) limits for a ratio of the geometric mean of logarithmic transformed AUC_{0-∞}, AUC₀₋ₜ, and Cₘₐₓ for the two products or two methods are about 0.80 to about 1.25.

In another embodiment, bioequivalence is determined according to the European Medicines Agency (EMEA) document “Note for Guidance on the Investigation of Bioavailability and Bioequivalence”, issued Jul. 26, 2001, available from EMEA.

To show bioequivalency between two compounds or administration conditions pursuant to Europe's EMEA guidelines, the 90% CI limits for a ratio of the geometric mean of logarithmic transformed AUC_{0-∞} and AUC₀₋ₜ for the two products or methods are about 0.80 to about 1.25. The 90% CI limits for a ratio of the geometric mean of logarithmic transformed Cₘₐₓ for the two products or methods can have a wider acceptance range when justified by safety and efficacy considerations. For example the acceptance range can be about 0.70 to about 1.43, specifically about 0.75 to about 1.33, and more specifically about 0.80 to about 1.25.

In one embodiment, in a given experiment, an active agent composition is considered to be bioequivalent to the reference drug if both the Test/Reference ratio for the geometric mean of logarithmic transformed AUC_{0-∞}, AUC₀₋ₜ, or Cₘₐₓ ratio along with its corresponding lower and upper 90% CI limits are within a lower limit of about 0.80 and an upper limit of about 1.25. Thus, for direct comparison between an inventive active agent composition and a reference drug, the pharmacokinetic parameters for the active agent composition and the reference drug can be determined in side-by side in the same pharmacokinetic study.

In some embodiments a single dose bioequivalence study is performed under non-fasted or fasted conditions.

In other embodiments, the single dose bioequivalence study is conducted between the active agent composition and the reference drug using the strength specified by the FDA in APPROVED DRUG PRODUCTS WITH THERAPEUTIC EQUIVALENCE EVALUATIONS(ORANGE BOOK).

In some embodiments, an in vivo bioequivalence study is performed to compare all active agent compositions with corresponding strengths of the reference drug (e.g., 500 or 750 mg of the active agent). In other embodiments, an in vivo bioequivalence study is performed only for the active agent composition of the present invention at the strength of the reference listed drug product (e.g., the highest approved strength) and at the other lower or higher strengths, the inventive compositions meet a reference drug dissolution test.

In one embodiment, an extended-release formulation comprises a matrix comprising levetiracetam or a pharmaceutically acceptable salt, solvate, hydrate, crystalline form or non-crystalline form thereof, and a hydrophobic excipient or an acrylic polymer excipient; wherein the extended-release formulation is substantially free of an extended-release coating, and wherein the formulation exhibits a ratio of a geometric mean of logarithmic transformed AUC_{0-∞} of the extended-release formulation to a geometric mean of logarithmic transformed AUC_{0-∞} of levetiracetam reference drug approved under the New Drug Application No. 022285 of about 0.80 to about 1.25 under fasting conditions or non-fasting condition.

In another embodiment, an extended-release formulation comprises a matrix comprising levetiracetam or a pharmaceutically acceptable salt, solvate, hydrate, crystalline form or non-crystalline form thereof, and a hydrophobic excipient or an acrylic polymer excipient; wherein the extended-release formulation is substantially free of an extended-release coating, and wherein the formulation exhibits a ratio of a geometric mean of logarithmic transformed AUC₀₋ₜ of the extended-release formulation to a geometric mean of logarithmic transformed AUC₀₋ₜ of levetiracetam reference drug approved under the New Drug Application No. 022285 of about 0.80 to about 1.25 under fasting conditions or non-fasting condition.

In yet another embodiment, an extended-release formulation comprises a matrix comprising levetiracetam or a pharmaceutically acceptable salt, solvate, hydrate, crystalline form or non-crystalline form thereof, and a hydrophobic excipient or an acrylic polymer excipient; wherein the extended-release formulation is substantially free of an extended-release coating, and wherein the formulation exhibits a ratio of a geometric mean of logarithmic transformed Cₘₐₓ of the extended-release formulation to a geometric mean of logarithmic transformed Cₘₐₓ of levetiracetam reference drug approved under the New Drug Application No. 022285 of about 0.70 to about 1.43 under fasting conditions or non-fasting condition.

In yet another embodiment, an extended-release formulation comprises a matrix comprising levetiracetam or a pharmaceutically acceptable salt, solvate, hydrate, crystalline form or non-crystalline form thereof, and a hydrophobic excipient or an acrylic polymer excipient; wherein the extended-release formulation is substantially free of an extended-release coating, and wherein the formulation exhibits a ratio of a geometric mean of logarithmic transformed Cₘₐₓ of the extended-release formulation to a geometric mean of logarithmic transformed Cₘₐₓ of levetiracetam reference drug approved under the New Drug Application No. 022285 of about 0.80 to about 1.25 under fasting conditions or non-fasting condition.

In one embodiment, an extended-release formulation comprises a matrix comprising levetiracetam or a pharmaceutically acceptable salt, solvate, hydrate, crystalline form or non-crystalline form thereof, and a hydrophobic excipient or an acrylic polymer excipient; wherein the extended-release formulation is substantially free of an extended-release coating, and wherein the formulation is bioequivalent to a reference drug according to New Drug Application No. 022285 (Keppra XR™, 500 milligrams) when administered to a patient in a fasted or non-fasted state.

In one embodiment, an extended-release formulation comprises a matrix comprising levetiracetam or a pharmaceutically acceptable salt, solvate, hydrate, crystalline form or non-crystalline form thereof, and a hydrophobic excipient or an acrylic polymer excipient; wherein the extended-release formulation is substantially free of an extended-release coating, and wherein the formulation exhibits substantially no food effect.

In another embodiment, an extended-release formulation comprises a matrix comprising levetiracetam or a pharmaceutically acceptable salt, solvate, hydrate, crystalline form or non-crystalline form thereof, and a hydrophobic excipient or an acrylic polymer excipient; wherein the extended-release formulation is substantially free of an extended-release coating, and wherein the formulation when administered to a patient in a non-fasted state is bioequivalent to the formulation when administered to a patient in a fasted state.

In still another embodiment, an extended-release formulation comprises a matrix comprising levetiracetam or a pharmaceutically acceptable salt, solvate, hydrate, crystalline form or non-crystalline form thereof, and a hydrophobic excipient or an acrylic polymer excipient; wherein the extended-release formulation is substantially free of an extended-release coating, and wherein the formulation exhibits a ratio of a geometric mean of logarithmic transformed AUC_{0-∞} of the formulation administered in a non-fasted state to a geometric mean of logarithmic transformed AUC_{0-∞} of the formulation administered in a fasted state of about 0.80 to about 1.25.

In one embodiment, an extended-release formulation comprises a matrix comprising levetiracetam or a pharmaceutically acceptable salt, solvate, hydrate, crystalline form or non-crystalline form thereof, and a hydrophobic excipient or an acrylic polymer excipient; wherein the extended-release formulation is substantially free of an extended-release coating, and wherein the formulation exhibits a ratio of a geometric mean of logarithmic transformed AUC₀₋ₜ of the formulation administered in a non-fasted state to a geometric mean of logarithmic transformed AUC₀₋ₜ of the formulation administered in a fasted state of about 0.80 to about 1.25.

In an embodiment, an extended-release formulation comprises a matrix comprising levetiracetam or a pharmaceutically acceptable salt, solvate, hydrate, crystalline form or non-crystalline form thereof, and a hydrophobic excipient or an acrylic polymer excipient; wherein the extended-release formulation is substantially free of an extended-release coating, and wherein the formulation exhibits a ratio of a geometric mean of logarithmic transformed Cₘₐₓ of the formulation administered in a non-fasted state to a geometric mean of logarithmic transformed geometric mean Cₘₐₓ of the formulation administered in a fasted state of about 0.80 to about 1.25.

The formulations disclosed herein comprise a matrix comprising an active agent, a hydrophobic excipient or an acrylic polymer excipient, and optionally additional excipients, specifically excluding a hydrophilic polymeric excipient.

The hydrophobic polymer excipient can include a wax excipient; cellulose ethers such as ethyl cellulose, methyl cellulose, and cellulose acetate; polyvinyl alcohol-maleic anhydride copolymers; and combinations thereof. In comparison, hydrophilic polymeric excipients include, for example, hydroxyethyl cellulose, hydroxypropyl cellulose, sodium alginate, carbomer (Carbopol®), sodium carboxymethyl cellulose, xanthan gum, guar gum, locust bean gum, poly vinyl acetate, polyvinyl alcohol, and hydroxypropyl methylcellulose.

The wax excipient for use in the matrix can be a solid wax at ambient temperature, such as a solid, hydrophobic material (i.e., non-water soluble) or solid hydrophilic material (e.g., polyethylene glycols are water soluble), but specifically a solid, hydrophobic material.

Exemplary wax excipients include wax and wax-like excipients, for example, carnauba wax (from the palm tree Copernicia Cerifera), vegetable wax, fruit wax, microcrystalline wax (“petroleum wax”), bees wax (white or bleached, and yellow), hydrocarbon wax, paraffin wax, cetyl esters wax, non-ionic emulsifying wax, anionic emulsifying wax, candelilla wax, or a combination comprising at least one of the foregoing waxes. Other suitable wax excipients include, for example, fatty alcohols (such as lauryl, myristyl, stearyl, cetyl or specifically cetostearyl alcohol), hydrogenated vegetable oil, hydrogenated castor oil, fatty acids such as stearic acid, fatty acid esters including fatty acid glycerides (mono-, di-, and tri-glycerides), polyethylene glycol (PEG) having a molecular weight of greater than about 3000 number average molecular weight, Mₙ, (e.g., PEG 3350, PEG 4000, PEG 4600, PEG 6000, and PEG 8000), or a combination comprising at least one of the foregoing wax excipients. Any combination of wax excipients is also contemplated.

In one embodiment, the wax excipient excludes polyethylene glycol.

The melting point of the wax excipient is a temperature above ambient temperature, specifically about 30 to about 150° C., more specifically about 75 to about 100° C., and yet more specifically about 75 to about 90° C.

Suitable acrylic polymers for use as a release-retarding material in the matrix include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, or a combination comprising at least one of the foregoing polymers. The acrylic polymer may comprise methacrylate copolymers described in NF XXIV as fully polymerized copolymers of acrylic and methacrylic acid esters with a low content of quaternary ammonium groups.

The amount of hydrophobic excipient or an acrylic polymer excipient present in the matrix can be determined based on the particular excipient or excipient combination chosen and the targeted release profile desired for the resulting formulation. Exemplary amounts of a hydrophobic excipient or an acrylic polymer excipient include about 5 to about 60 wt. % based on the total weight of the matrix of the extended-release formulation, specifically about 15 to about 50 wt. %, more specifically about 20 to about 42 wt. %, yet more specifically about 25 to about 35 wt. % and still yet more specifically about 27 to about 30 wt. % based on the total weight of the matrix of the extended-release formulation.

In another embodiment, the hydrophobic excipient is a combination of carnauba wax and stearic acid in a weight ratio of about 1.5:1 to about 2.5:1 carnauba wax:stearic acid, specifically about 1.75:1 to about 2.25:1, more specifically about 1.9:1 to about 2.1:1, and still more specifically about 2:1.

In another embodiment, the hydrophobic excipient is a combination of carnauba wax and stearic acid, the carnauba wax is present about 10 to about 30 wt. % based on the total weight of the matrix of the extended-release formulation, specifically about 15 to about 25 wt. %, more specifically about 17 to about 20 wt. %, and yet more specifically about 17.5 to about 19.5 wt. %; and the stearic acid is present at about 5 to about 15 wt. % based on the total weight of the matrix of the extended-release formulation, specifically about 8 to about 12 wt. %, more specifically about 9 to about 11 wt. %, and yet more specifically about 9.5 to about 10.5 wt. %.

In one embodiment, the matrix comprises levetiracetam in an amount of about 60 to about 98 wt. % based on the total weight of the matrix of the extended-release formulation, specifically about 65 to about 90 wt. %, more specifically about 68 to about 85 wt. %, yet more specifically about 70 to about 80 wt. %, and still more specifically about 72 to about 75 wt. %.

In one embodiment, the formulation can contain about 250 mg to about 1.5 grams of levetiracetam, specifically about 500 mg to about 1.0 gram, and more specifically about 750 mg per unit. In one embodiment, the formulation is a tablet containing about 500 to about 750 mg of levetiracetam per tablet.

In an embodiment, the levetiracetam formulation comprises a matrix that is substantially free of or free of a hydrophilic polymeric excipient. As used herein, hydrophilic polymeric excipients include hydroxylated cellulosic binders (e.g., hydroxypropylmethyl cellulose, hydroxypropyl cellulose, hydroxyethyl cellulose, and the like), polyvinylpyrrolidone, starch, pregelatinized starch, modified corn starch, polyacryl amide, poly-N-vinyl amide, sodium carboxymethyl cellulose, gelatin, polyethylene oxide, poly propylene glycol, tragacanth, alginic acid, sodium alginate, carbomer, xanthan gum, guar gum, locust bean gum, polyvinyl acetate, polyvinyl alcohol and the like; and the term specifically excludes excipients such as glidants and lubricants. As used herein, “substantially free of a hydrophilic polymeric excipient” means the matrix contains less than 1 wt. % hydrophilic polymeric excipient, specifically less than 0.5% hydrophilic polymeric excipient, and more specifically 0 wt. % hydrophilic polymeric excipient based on the total weight of the matrix of the extended-release formulation.

In another embodiment, the matrix optionally further contains a hydrophilic polymeric excipient as an additional release-retarding material.

The hydrophilic polymeric excipient can be present in the matrix of the extended-release formulation in an amount of 0 to about 65 wt. % based on the total weight of the matrix of the extended-release formulation, specifically about 0.1 to about 50 wt. %, more specifically about 10 to about 45 wt. %, and yet more specifically about 15 to about 30 wt. %. Besides the additional release-retarding material, the additional excipients optionally include fillers, disintegrants, lubricants, glidants, and the like.

The optional disintegrant is used to facilitate the breakdown of the extended-release formulation in a fluid environment, specifically aqueous environments. The choice and amount of disintegrant is tailored to ensure the desired dissolution profile of the formulation or to provide the desired controlled-release in vivo. Exemplary disintegrants include a material that possesses the ability to swell or expand upon exposure to a fluid environment, especially an aqueous environment. Exemplary disintegrants include hydroxyl substituted alkyl celluloses (e.g., hydroxypropyl cellulose), starch, pregelatinized starch (e.g., Starch 1500® available from Colorcon); cross-linked sodium carboxymethylcellulose (e.g., “croscarmellose sodium”, i.e., Ac-Di-Sol® available from FMC BioPolymer of Philadelphia, Pa.); crosslinked homopolymer of N-vinyl-2-pyrrolidone (e.g., “crospovidone”, e.g., Polyplasdone® XL, Polyplasdone® XL-10, and Polyplasdone® INF-10 available from International Specialty Products, Wayne N.J.); modified starches, such as sodium carboxymethyl starch, sodium starch glycolate (e.g., Primogel®), and the like; alginates; or a combination comprising at least one of the foregoing disintegrants.

The amount of disintegrant used depends upon the disintegrant or disintegrant combination chosen and the targeted release profile of the resulting formulation. Exemplary amounts include about 0 to about 10 wt. % based on the total weight of the matrix of the extended-release formulation, specifically about 0.1 to about 7.0 wt. %, and yet more specifically about 1.0 to about 5.0 wt. %.

Exemplary lubricants include stearates (e.g., calcium stearate, magnesium stearate, and zinc stearate), sodium stearyl fumarate, mineral oil, talc, or a combination comprising at least one of the foregoing. Glidants include, for example, silicon dioxide (e.g., fumed or colloidal). It is recognized that certain materials can function both as a glidant and a lubricant.

The lubricant or glidant is used in amounts of about 0.1 to about 15 wt. % of the total weight of the extended-release formulation; specifically about 0.5 to about 5 wt. %; and yet more specifically about 0.75 to about 3 wt. %.

The extended-release formulations are prepared by processes known in the art, including granulation (dry or wet) and compression, spheronization, melt extrusion, hot fusion, and the like.

Once the extended-release formulation is formed, it can optionally be coated with a non-functional coating. By “functional coating” is meant to include a coating that modifies the release properties of the total formulation, for example, a controlled-release coating that provides extended-release of the active agent. By “non-functional coating” is meant to include a coating that does not significantly modify the release properties of the total formulation, for example, a cosmetic coating or for identification purposes. A non-functional coating can have some impact on the release of the active agent due to the initial dissolution, hydration, perforation of the coating, and the like, but would not be considered to be a significant deviation from the non-coated composition.

In one embodiment, an extended-release formulation comprises a matrix comprising levetiracetam or a pharmaceutically acceptable salt, solvate, hydrate, crystalline form or non-crystalline form thereof, and a hydrophobic excipient; wherein the extended-release formulation is substantially free of an extended-release coating, and wherein the formulation exhibits a dissolution profile such that at one hour after combining the formulation with 900 ml of deionized water, 0.1 N HCl, pH 4.5 acetate buffer, or pH 6.8 potassium phosphate buffer at 37° C.±0.5° C. according to USP 28 <711> test method 2 (paddle), 75 rpm paddle speed, using Japanese sinkers, about 30 to about 50 wt. % of the total amount of active agent is released.

In another embodiment, an extended-release formulation comprises a matrix comprising levetiracetam or a pharmaceutically acceptable salt, solvate, hydrate, crystalline form or non-crystalline form thereof, and a hydrophobic excipient; wherein the extended-release formulation is substantially free of an extended-release coating, and wherein the formulation exhibits a dissolution profile such that two hours after combining the formulation with 900 ml of deionized water, 0.1 N HCl, pH 4.5 acetate buffer, or pH 6.8 potassium phosphate buffer at 37° C.±0.5° C. according to USP 28 <711> test method 2 (paddle), 75 rpm paddle speed, using Japanese sinkers, about 35 to about 65 wt. % of the total amount of active agent is released.

In yet another embodiment, an extended-release formulation comprises a matrix comprising levetiracetam or a pharmaceutically acceptable salt, solvate, hydrate, crystalline form or non-crystalline form thereof, and a hydrophobic excipient; wherein the extended-release formulation is substantially free of an extended-release coating, and wherein the formulation exhibits a dissolution profile such that four hours after combining the formulation with 900 ml of deionized water, 0.1 N HCl, pH 4.5 acetate buffer, or pH 6.8 potassium phosphate buffer at 37° C.±0.5° C. according to USP 28 <711> test method 2 (paddle), 75 rpm paddle speed, using Japanese sinkers, about 50 to about 85 wt. % of the total amount of active agent is released.

In still another embodiment, an extended-release formulation comprises a matrix comprising levetiracetam or a pharmaceutically acceptable salt, solvate, hydrate, crystalline form or non-crystalline form thereof, and a hydrophobic excipient; wherein the extended-release formulation is substantially free of an extended-release coating, and wherein the formulation exhibits a dissolution profile such that eight hours after combining the formulation with 900 ml of deionized water, 0.1 N HCl, pH 4.5 acetate buffer, or pH 6.8 potassium phosphate buffer at 37° C.±0.5° C. according to USP 28 <711> test method 2 (paddle), 75 rpm paddle speed, using Japanese sinkers, about 75 to about 100 wt. % of the total amount of active agent is released.

In another embodiment, an extended-release formulation comprises a matrix comprising levetiracetam or a pharmaceutically acceptable salt, solvate, hydrate, crystalline form or non-crystalline form thereof, and a hydrophobic excipient; wherein the extended-release formulation is substantially free of an extended-release coating, and wherein the formulation exhibits a dissolution profile such that twelve hours after combining the formulation with 900 ml of deionized water, 0.1 N HCl, pH 4.5 acetate buffer, or pH 6.8 potassium phosphate buffer at 37° C.±0.5° C. according to USP 28 <711> test method 2 (paddle), 75 rpm paddle speed, using Japanese sinkers, about 85 to about 100 wt. % of the total amount of active agent is released.

Also included herein are pharmaceutical kits which comprise one or more containers containing a controlled-release formulation as described herein. The kits may further comprise one or more conventional pharmaceutical kit components, such as, for example, one or more containers to aid in facilitating compliance with a particular dosage regimen; one or more carriers; printed instructions, either as inserts or as labels, indicating quantities of the components to be administered, or guidelines for administration. Exemplary kits can be in the form of bubble or blister pack cards, optionally arranged in a desired order for a particular dosing regimen. Suitable blister packs that can be arranged in a variety of configurations to accommodate a particular dosing regimen are well known in the art or easily ascertained by one of ordinary skill in the art.

In one embodiment, a method of treating a patient comprises administering an extended-release formulation to a patient in need thereof, wherein the formulation comprises a matrix comprising levetiracetam or a pharmaceutically acceptable salt, solvate, hydrate, crystalline form or non-crystalline form thereof, and a hydrophobic excipient; wherein the extended-release formulation is substantially free of an extended-release coating. The patient may be treated for epilepsy, neuropathic pain, seizures, and the like.

### EXAMPLES

### Example 1

### Preparation of Levetiracetam Extended-Release Tablets, 500 Mg and 750 Mg

Extended-release levetiracetam tablets are prepared having the components listed in Table 1 below.

TABLE 1

| | A, 500 mg | | B, 750 mg | | C, 750 mg | |
|---|---|---|---|---|---|---|
| | Weight | % of | Weight | % of | Weight | % of |
| Component | (mg/tablet) | tablet | (mg/tablet) | tablet | (mg/tablet) | tablet |
| Levetiracetam | 500.0 | 68.49 | 750 | 70.7547 | 750 | 68.5 |
| Carnauba Wax | 140.0 | 19.18 | 190 | 17.9245 | 210 | 19.2 |
| Stearic acid | 75.0 | 10.27 | 100 | 9.434 | 112.5 | 10.3 |
| Denatured | 50 microliters | — | 75 microliters | — | 75 microliters | — |
| alcohol* | | | | | | |
| Silicon dioxide | 7.5 | 1.03 | 10 | 0.9434 | 11.25 | 1.0 |
| (Syloid 244 FP) | | | | | | |
| Magnesium | 7.5 | 1.03 | 10 | 0.9434 | 11.25 | 1.0 |
| Stearate | | | | | | |
| Total | 730 | 100 | 1060 | 100 | 1095 | 100 |

*Not present in final product

The tablets are prepared by dissolving stearic acid in the denatured alcohol with mixing and gentle heat (˜50° C.). Levetiracetam and carnauba wax are screened and mixed in a mixer/granulator. The stearic acid mixture is added to the active agent/wax mixture and granulated to form granules. The resulting granules are dried and milled. The milled granules are charged to a Gemco Blender to which screened silicon dioxide is added and mixed. Screened magnesium stearate is then added and mixed to form a blend. The resulting blend is then compressed into extended-release tablets.

The extended-release tablets are then coated with a non-functional film coating solution to achieve a targeted weight gain of about 2% (about 22 mg film coating per 500 mg tablet or 33 mg per 750 mg tablet) using Opadry II, a hydroxypropyl methylcellulose non-functional coating.

### Example 2

### Comparative Dissolution Between Keppra XR™ and the Formulations of Example 1

A comparison of in vitro dissolution was conducted between 500 mg tablet KEPPRA XR™ and the extended-release levetiracetam tablet A of Example 1 using the test method protocol according to USP 26, 711, 900 milliliters of deionized (DI) water, 0.1 N HCl, pH 4.5 acetate buffer, or pH 6.8 potassium phosphate buffer at 37° C.±0.5° C. and a paddle speed of 75 rotations per minute (rpm) with Japanese sinkers. The results of the dissolution analyses are summarized in Table 2; each data point is an average of six samples.

TABLE 2

| | | | | | | | Keppra XR, | Keppra XR, |
|---|---|---|---|---|---|---|---|---|
| | Ex. 1A | Ex. 1A | Ex. 1A | Ex. 1A | Keppra XR, | Keppra XR, | 500 mg | 500 mg |
| | 0.1 N | DI | pH 4.5 | pH 6.8 | 500 mg | 500 mg | pH 4.5 | pH 6.8 |
| Time | HCl | water | buffer | buffer | 0.1 N HCl | DI water | buffer | buffer |
| (hr) | % dissolved | | | | | | | |
| 0 | 0 | 0 | 0 | 0 | 0 | 0 | 0 | 0 |
| 1 | 40 | 40 | 41 | 42 | 28 | 33 | 33 | 33 |
| 2 | 56 | 57 | 57 | 57 | 46 | 51 | 51 | 52 |
| 3 | 67 | 69 | 69 | 68 | 59 | 65 | 65 | 64 |
| 4 | 74 | 78 | 78 | 76 | 70 | 76 | 75 | 74 |
| 6 | 85 | 88 | 89 | 86 | 83 | 91 | 90 | 88 |
| 8 | 92 | 96 | 97 | 94 | 90 | 100 | 98 | 97 |
| 10 | 96 | 100 | 101 | 99 | 93 | 104 | 101 | 100 |
| 12 | 98 | 102 | 102 | 101 | 94 | 106 | 103 | 102 |
| 18 | — | — | — | — | 93 | 108 | 104 | 103 |
| 24 | — | — | — | — | 93 | 108 | 105 | 104 |

As the dissolution results in Table 2 indicate, the extended-release levetiracetam tablets provide a long, controlled delivery of levetiracetam exhibiting substantially the same release profile as KEPPRA XR™ over the hours of 2 to 12.

A comparison of in vitro dissolution was conducted between the 750 mg tablet KEPPRA XR™ and the extended-release levetiracetam tablet of Example 1C using the test method protocol according to USP 26, 711, 900 milliliters of deionized (DI) water, 0.1 N HCl, pH 4.5 acetate buffer, or pH 6.8 potassium phosphate buffer at 37° C.±0.5° C. and a paddle speed of 75 rotations per minute (rpm) with Japanese sinkers. The results of the dissolution analyses are summarized in Table 3; each data point is an average of twelve samples.

TABLE 3

| | | | | | | | Keppra XR, | Keppra XR, |
|---|---|---|---|---|---|---|---|---|
| | Ex. 1C | Ex. 1C | Ex. 1C | Ex. 1C | Keppra XR, | Keppra XR, | 750 mg | 750 mg |
| | 0.1 N | DI | pH 4.5 | pH 6.8 | 750 mg | 750 mg | pH 4.5 | pH 6.8 |
| Time | HCl | water | buffer | buffer | 0.1 N HCl | DI water | buffer | buffer |
| (hr) | % dissolved | | | | | | | |
| 0 | 0 | 0 | 0 | 0 | 0 | 0 | 0 | 0 |
| 1 | 35 | 37 | 36 | 35 | 28 | 28 | 28 | 27 |
| 2 | 48 | 50 | 49 | 49 | 42 | 42 | 42 | 42 |
| 4 | 63 | 67 | 66 | 65 | 62 | 62 | 63 | 62 |
| 8 | 80 | 85 | 85 | 83 | 85 | 86 | 87 | 85 |
| 12 | 89 | 94 | 94 | 93 | 95 | 96 | 97 | 96 |
| 16 | 93 | — | 98 | 98 | 97 | — | 101 | 100 |
| 18 | — | 98 | — | — | — | 100 | — | — |
| 24 | — | 99 | — | — | — | 100 | — | — |

As the dissolution results in Table 3 indicate, the extended-release levetiracetam tablet of Example 1C provides a long, controlled delivery of levetiracetam exhibiting substantially the same release profile as KEPPRA XR™ 750 mg.

### Example 3

### Relative Bioavailability Under Fasting Conditions of the Extended-Release Tablet Formulation of Example 1A in Comparison to Keppra XR™ Tablet

A 2-arm, open-label, single-dose, fasted relative bioavailability study of the levetiracetam extended-release formulation of Example 1A versus 500 mg KEPPRA XR™ tablet reference (“Reference”) is performed in healthy, adult volunteers. The study is performed on 29 subjects. Each subject participates in two dosing periods separated by a washout period of at least seven days. The two dosing regimens are one 500 mg tablet of Example 1A (test product), and one 500 mg KEPPRA XR™ tablet (Reference) preceded by an overnight fast of at least 10 hours. Subjects are confined at the early evening prior to and until at least 24 hours after dosing. Blood samples are drawn from each subject for drug content analysis at time zero (predose) and after dose administration every ½ hour for the first eight hours, then at hours 9, 10, 12, 16, 20, 24, 36, 48, and 72. Levetiracetam plasma concentrations in the blood samples are measured using a validated bioanalytical method.

The levetiracetam concentration-time data are used to calculate the following pharmacokinetic parameters: AUC₀₋ₜ, AUC_{0-∞}, Cₘₐₓ, Tₘₐₓ, kₑ, and t_{1/2}. The pharmacokinetic parameters are evaluated statistically by an analysis of variance (ANOVA) appropriate for the experimental design of the study. Analyses for AUC₀₋ₜ, AUC_{0-∞}, and Cₘₐₓ are performed on 1n-transformed data. For 1n-transformed AUC₀₋ₜ, AUC_{0-∞}, and Cₘₐₓ, estimates of the adjusted differences between treatment means and the standard error associated with these differences are used to construct a 90% confidence interval for the ratio of the test to reference population means.

TABLE 4
Formulation Example 1A versus
KEPPRA XR ™ 500 mg, Fasting, N = 29

| | | | | |
|---|---|---|---|---|
| | | | | 90% |
| | | | | Confidence |
| | | Reference | | Interval |
| | Formulation | [KEPPRA | % | (Lower Limit, |
| PK variable | Example 1A | XR ™ 500 mg] | Ratio | Upper Limit) |

Ln-transformed data

| | Geometric Mean | | | |
|---|---|---|---|---|
| Cₘₐₓ (ng/ml) | 7.72 | 7.64 | 101.03 | (97.32, 104.88) |
| AUC₀₋ₜ | 120.04 | 122.17 | 98.26 | (95.1, 101.51) |
| (ng-hr/ml) | | | | |
| AUC_{0-INF} | 134.04 | 133.97 | 100.05 | (97.68, 102.48) |
| (ng-hr/ml) | | | | |

Non-transformed data

| | least squares mean | | | |
|---|---|---|---|---|
| Cmax (ng/ml) | 7.86 | 7.81 | 100.60 | (97.04, 104.15) |
| AUC₀₋ₜ | 122.82 | 124.85 | 98.38 | (95.36, 101.4) |
| (ng-hr/ml) | | | | |
| AUC_{0-INF} | 136.60 | 135.99 | 100.45 | (97.99, 102.9) |
| (ng-hr/ml) | | | | |
| Tmax | 3.50 | 4.50 | 77.78 | (66.83, 88.73) |
| kₑₗᵢₘ | 0.0791 | 0.0823 | 96.07 | (90.61, 101.53) |
| t_{1/2} | 9.00 | 8.60 | 104.57 | (97.86, 111.29) |

As the results in Table 4 indicate, the Formulation of Example 1A is bioequivalent to KEPPRA XR™ 500 mg under fasting conditions (90% confidence interval of 80-125% for AUC and Cₘₐₓ.

### Example 4

### Relative Bioavailability Under Non-Fasting Conditions of the Extended-Release Tablet Formulation of Example 1A in Comparison to Keppra XR™ Tablet

A similar bioavailability study is performed as described in Example 3 although the test and reference tablets are administered to the subjects within five minutes of consuming an entire standard high-fat breakfast. The data are analyzed as previously described in Example 3.

TABLE 5
Formulation Example 1A versus
KEPPRA XR ™ 500 mg, Non-Fasting, N = 30

| | | | | |
|---|---|---|---|---|
| | | | | 90% |
| | | | | Confidence |
| | | Reference | | Interval |
| | Formulation | [KEPPRA | % | (Lower Limit, |
| PK variable | Example 1A | XR ™ 500 mg] | Ratio | Upper Limit) |

Ln-transformed data

| | Geometric Mean | | | |
|---|---|---|---|---|
| Cₘₐₓ (ng/ml) | 8.09 | 7.98 | 101.46 | (97.73, 105.34) |
| AUC₀₋ₜ | 113.76 | 115.22 | 98.73 | (96.24, 101.28) |
| (ng-hr/ml) | | | | |
| AUC_{0-INF} | 128.08 | 128.62 | 99.58 | (98.27, 100.91) |
| (ng-hr/ml) | | | | |

Non-transformed data

| | least squares mean | | | |
|---|---|---|---|---|
| Cmax (ng/ml) | 8.19 | 8.10 | 101.14 | (97.07, 105.21) |
| AUC₀₋ₜ | 115.21 | 116.73 | 98.70 | (96.25, 101.14) |
| (ng-hr/ml) | | | | |
| AUC_{0-INF} | 129.10 | 129.66 | 99.57 | (98.22, 100.91) |
| (ng-hr/ml) | | | | |
| Tmax | 4.67 | 5.65 | 82.60 | (73.85, 91.34) |
| kₑₗᵢₘ | 0.0875 | 0.0896 | 97.69 | (93.75, 101.62) |
| t_{1/2} | 8.10 | 7.86 | 103.05 | (98.52, 107.58) |

As the results in Table 5 indicate, the Formulation of Example 1A is bioequivalent to KEPPRA XR™ 500 mg under non-fasting conditions (90% confidence interval of 80-125% for AUC and Cₘₐₓ).

In a separate food effect study of KEPPRA XR™ 500 mg (N=15), the results indicate that the bioavailability of KEPPRA XR™ 500 mg administered under non-fasting conditions is bioequivalent to the results under fasting conditions (90% confidence interval of 80-125% for AUC and Cₘₐₓ) thus confirming the brand tablet exhibits no food effect. Likewise, in view of the results of Tables 4-5, the formulation of Example 1A is expected to exhibit no food effect.

### Example 5

### Relative Bioavailability Under Fasting Conditions of the 750 Mg Extended-Release Tablet Formulation of Example 1C in Comparison to Keppra XR™ Tablet

A similar bioavailability study is performed as described in Example 4 although the test tablet is Formulation Example 1C of Example 1 and the reference tablet is Keppra XR™ 750 mg (“Reference”). The data are analyzed as previously described in Example 4.

TABLE 6
Formulation Example 1C versus
KEPPRA XR ™ 750 mg, Fasting, N = 28

| | | | | |
|---|---|---|---|---|
| | | | | 90% |
| | | | | Confidence |
| | | Reference | | Interval |
| | Formulation | [KEPPRA | % | (Lower Limit, |
| PK variable | Example 1C | XR ™ 750 mg] | Ratio | Upper Limit) |

Ln-transformed data

| | Geometric Mean | | | |
|---|---|---|---|---|
| Cₘₐₓ (ng/ml) | 9.34 | 9.14 | 102.26 | (98.83, 105.81) |
| AUC₀₋ₜ | 166.26 | 172.94 | 96.14 | (92.23, 100.22) |
| (ng-hr/ml) | | | | |
| AUC_{0-INF} | 178.32 | 183.37 | 97.24 | (94.05, 100.55) |
| (ng-hr/ml) | | | | |

Non-transformed data

| | least squares mean | | | |
|---|---|---|---|---|
| Cmax (ng/ml) | 9.63 | 9.44 | 102.00 | (98.26, 105.73) |
| AUC₀₋ₜ | 170.40 | 176.25 | 96.68 | (93.59, 99.77) |
| (ng-hr/ml) | | | | |
| AUC_{0-INF} | 181.80 | 186.45 | 97.51 | (94.9, 100.11) |
| (ng-hr/ml) | | | | |
| Tmax | 3.71 | 4.71 | 78.79 | (64.11, 93.47) |

As the results in Table 6 indicate, the Formulation of Example 1C is bioequivalent to KEPPRA XR™ 750 mg under fasting conditions (90% confidence interval of 80-125% for AUC and C.

### Example 6

### Relative Bioavailability Under Non-Fasting Conditions of the Extended-Release Tablet Formulation of Example 1C in Comparison to Keppra XR™ Tablet

A similar bioavailability study is performed as described in Example 4 although the test tablet is Formulation Example 1C of Example 1 and the reference tablet is Keppra XR™ 750 mg (“Reference”). The data are analyzed as previously described in Example 3.

TABLE 7
Formulation Example 1 versus
KEPPRA XR ™ 500 mg, Non-Fasting, N = 30

| | | | | |
|---|---|---|---|---|
| | | | | 90% Confidence |
| | | Reference | | Interval |
| | Formulation | [KEPPRA | % | (Lower Limit, |
| PK variable | Example 1C | XR ™ 750 mg] | Ratio | Upper Limit) |

Ln-transformed data

| | Geometric Mean | | | |
|---|---|---|---|---|
| Cₘₐₓ (ng/ml) | 8.84 | 9.92 | 89.19 | (86.45, 92.01) |
| AUC₀₋ₜ | 167.12 | 170.22 | 98.18 | (95.86, 100.55) |
| (ng-hr/ml) | | | | |
| AUC_{0-INF} | 177.04 | 180.47 | 98.10 | (96.26, 99.97) |
| (ng-hr/ml) | | | | |

Non-transformed data

| | least squares mean | | | |
|---|---|---|---|---|
| Cmax (ng/ml) | 9.02 | 10.14 | 88.95 | (85.77, 92.14) |
| AUC₀₋ₜ | 169.95 | 172.79 | 98.35 | (96.01, 100.7) |
| (ng-hr/ml) | | | | |
| AUC_{0-INF} | 179.67 | 183.16 | 98.06 | (96.24, 99.95) |
| (ng-hr/ml) | | | | |
| Tmax | 5.47 | 6.50 | 84.10 | (73.76, 94.45) |

As the results in Table 7 indicate, the Formulation of Example 1C is bioequivalent to KEPPRA XR™ 750 mg under non-fasting conditions (90% confidence interval of 80-125% for AUC and Cₘₐₓ).

In all embodiments disclosed herein which are directed to extended-release formulations substantially free of an extended-release coating, the corresponding embodiments to extended-release formulations free of an extended-release coating are also included.

In all embodiments disclosed herein which are directed to extended-release formulations wherein the matrix is substantially free of a hydrophilic polymeric excipient, the corresponding embodiments to extended-release formulations wherein the matrix is free of a hydrophilic polymeric excipient are also included.

In all embodiments disclosed herein which are directed to extended-release formulations exhibiting substantially no food effect, the corresponding embodiments to extended-release formulations exhibiting no food effect are also included.

Embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. An extended-release formulation, comprising:
a matrix comprising
levetiracetam or a pharmaceutically acceptable salt, solvate, hydrate, crystalline form or non-crystalline form thereof, and
a hydrophobic excipient or an acrylic polymer excipient;
wherein the extended-release formulation is substantially free of an extended-release coating.

2. The formulation of claim 1, wherein the hydrophobic excipient is carnauba wax, vegetable wax, fruit wax, microcrystalline wax, bees wax, hydrocarbon wax, paraffin wax, cetyl esters wax, non-ionic emulsifying wax, anionic emulsifying wax, candelilla wax, stearyl alcohol, cetyl alcohol, cetostearyl alcohol, lauryl alcohol, myristyl alcohol, a hydrogenated vegetable oil, a hydrogenated castor oil, a fatty acid, a fatty acid ester, a fatty acid glyceride, a polyethylene glycol having a Mₙ, of greater than about 3000, or a combination comprising at least one of the foregoing wax excipients.

3. The formulation of claim 1, wherein the hydrophobic excipient is a combination of carnauba wax and stearic acid.

4. The formulation of claim 1, wherein the hydrophobic excipient or an acrylic polymer excipient is present in an amount of about 15 to about 50 wt. % based on the total weight of the extended-release formulation.

5. The formulation of claim 1, wherein the hydrophobic excipient or an acrylic polymer excipient is present in an amount of about 20 to about 42 wt. % based on the total weight of the extended-release formulation.

6. The formulation of claim 1, wherein the hydrophobic excipient or an acrylic polymer excipient is present in an amount of about 25 to about 35 wt. % based on the total weight of the extended-release formulation

7. The formulation of claim 1, wherein the extended-release formulation is prepared by wet granulation and compression processes.

8. The formulation of claim 1, wherein the matrix is substantially free of a hydrophilic polymeric excipient.

9. The formulation of claim 1, wherein the matrix is free of a hydrophilic polymeric excipient.

10. The formulation of claim 1, wherein the extended-release formulation is free of an extended-release coating.

11. The formulation of claim 1, wherein the extended-release formulation is coated with a non-functional coating.

12. The formulation of claim 1, wherein the extended-release formulation exhibits a dissolution profile such that at one hour after combining the formulation with 900 ml of deionized water, 0.1 N HCl, pH 4.5 acetate buffer, or pH 6.8 potassium phosphate buffer at 37° C.±0.5° C. when tested using a tablet dissolution apparatus equipped with a paddle stirring element, 75 rpm paddle speed with Japanese sinkers, about 30 to about 50 wt. % of the total amount of active agent is released.

13. The formulation of claim 12, wherein after two hours, about 35 to about 65 wt. % of the total amount of the active agent is released.

14. The formulation of claim 13, wherein after four hours, about 50 to about 85 wt. % of the total amount of the active agent is released.

15. The formulation of claim 14, wherein after eight hours about 75 to about 100 wt. % of the total amount of the active agent is released.

16. The formulation of claim 15, wherein after twelve hours about 85 to about 100 wt. % of the total amount of the active agent is released.

17. The formulation of claim 1, wherein the extended-release formulation is bioequivalent to a reference drug according to New Drug Application No. 022285 when administered to a patient in a fasted or non-fasted state.

18. The formulation of claim 17, wherein the dosage strength is about 500 mg, about 750, about 1000 mg, or about 1500 mg levetiracetam.

19. The formulation of claim 1, wherein the extended-release formulation exhibits
a ratio of a geometric mean of logarithmic transformed AUC_{0-∞} of the extended-release formulation to a geometric mean of logarithmic transformed AUC_{0-∞} of reference drug (New Drug Application No. 022285) of about 0.80 to about 1.25;
a ratio of a geometric mean of logarithmic transformed AUC₀₋ₜ of the extended-release formulation to a geometric mean of logarithmic transformed AUC₀₋ₜ of reference drug (New Drug Application No. 022285) of about 0.80 to about 1.25;
a ratio of a geometric mean of logarithmic transformed Cₘₐₓ of the extended-release formulation to a geometric mean of logarithmic transformed Cₘₐₓ of reference drug (New Drug Application No. 022285) of about 0.70 to about 1.43; or
a ratio of a geometric mean of logarithmic transformed Cₘₐₓ of the extended-release formulation to a geometric mean of logarithmic transformed Cₘₐₓ of reference drug (New Drug Application No. 022285) of about 0.80 to about 1.25,
wherein the foregoing are determined under fasting or non-fasting conditions.

20. The formulation of claim 1, wherein the extended-release formulation exhibits substantially no food effect.

21. The formulation of claim 1, wherein the extended-release formulation when administered to a patient in a non-fasted state is bioequivalent to the extended-release formulation when administered to a patient in a fasted state.

22. The formulation of claim 1, wherein the extended-release formulation exhibits a ratio of a geometric mean of logarithmic transformed AUC_{0-∞} of the extended-release formulation administered in a non-fasted state to a geometric mean of logarithmic transformed AUC_{0-∞} of the extended-release formulation administered in a fasted state of about 0.80 to about 1.25;
wherein the extended-release formulation exhibits a ratio of a geometric mean of logarithmic transformed AUC₀₋ₜ of the extended-release formulation administered in a non-fasted state to a geometric mean of logarithmic transformed AUC₀₋ₜ of the extended-release formulation administered in a fasted state of about 0.80 to about 1.25; or
wherein the extended-release formulation exhibits a ratio of a geometric mean of logarithmic transformed Cₘₐₓ of the extended-release formulation administered in a non-fasted state to a geometric mean of logarithmic transformed geometric mean Cₘₐₓ of the extended-release formulation administered in a fasted state of about 0.80 to about 1.25.

23. An extended-release formulation, comprising:
a matrix comprising
levetiracetam or a pharmaceutically acceptable salt, solvate, hydrate, crystalline form or non-crystalline form thereof,
about 15 to about 25 weight percent carnauba wax based on the total weight of the matrix, and
about 5 to about 15 weight percent stearic acid based on the total weight of the matrix;
wherein the extended-release formulation is substantially free of an extended-release coating.

24. A method of treating a patient, comprising
administering the extended-release formulation of claim 1 to a patient.
